(19) United States
(12) Patent Application Publication
(10) Pub. No.: US 2019/0192019 A1
(43) Pub. Date: Jun. 27, 2019

(54) BIOSENSOR AND SENSING METHOD USING THE SAME

(71) Applicant: SNU R&DB FOUNDATION, Seoul (KR)

(72) Inventors: Young-june PARK, Seoul (KR); Jun-myung WOO, Seoul (KR); Seok-hyang KIM, Seoul (KR); Jin-hong AHN, Yongin-si (KR)

(73) Assignee: SNU R&DB FOUNDATION, Seoul (KR)

(21) Appl. No.: 16/282,720

(22) Filed: Feb. 22, 2019

Related U.S. Application Data
(63) Continuation of application No. 13/947,483, filed on Jul. 22, 2013, now abandoned.

(30) Foreign Application Priority Data
Jul. 24, 2012 (KR) 10-2012-0080811
Oct. 25, 2012 (KR) 10-2012-0118887

(51) Int. Cl. A61B 5/04; G01N 27/414

(52) U.S. Cl. CPC A61B 5/04; A61B 5/1486; G01N 27/4145

(57) ABSTRACT

A biosensor including: a first electrode; a second electrode spaced from the first electrode; a channel unit electrically connected at a portion with the first electrode and electrically connected at another portion with the second electrode; a stimuli source electrically connected with the channel unit and applying an electric stimulus; and probes connected to the channel unit and complementarily bound with target materials to sense.

### CROSS REFERENCE TO PRIOR APPLICATIONS

This Application is a Continuation of U.S. patent application Ser. No. 13/947,483 filed on Jul. 22, 2013, which claims priority under 35 U.S.C. § 119 to Korean Patent Application Nos. 10-2012-0080811 filed on Jul. 24, 2012 and 10-2012-0118887 filed on Oct. 25, 2012, which are hereby incorporated by reference in their entirety.

### BACKGROUND

The present invention relates to a biosensor, a sensing method using the same, binding enhancing apparatus and a method of binding enhancing using the same.

Biosensors, which are sensors that can detect whether there is a biomaterial such as an enzyme, an antibody, an antigen, a protein, a hormone, and DNA and the concentration of the biomaterial, are available for various industrial fields, including medical field, medicine manufacture, environment, agriculture, and food.

The biosensors of the related art analyze a material to sense (target material) by reacting the component containing the target material with a bio receptor (probe) and then sensing the phenomenon due to the reaction with naked eyes or other various ways such as electro-chemical and optical methods.

### SUMMARY

Affinity-based biosensors are configured to have a probe, which is a material that is complementarily bound with a charged material, and to obtain data by sensing charge induced on a substrate by the charge of a target material and then processing signals by means of an analog/digital circuit, when the target material and the probe are bound.

When a target material with low fluidity or concentration is sensed, the sensing sensitivity of the target material is low because the binding rate of the probe and the target material per unit time is low. Further, when a target material contained in an electrolyte such as a buffer solution is sensed, a material having charge opposite to the target material surrounds the target material. Accordingly, the charge of the target material turns out being offset by the opposite charge, when observed at a distance from the target material, which is called a charge screen effect. The amount of charge that is induced on a substrate by the charge screen effect decreases, as compared with when there is no screen effect, such that sensing sensitivity decreases.

An object of the present invention is to provide an affinity-based biosensor with an improved sensing sensitivity. Another object of the present invention is to provide a measuring method that can improve sensing sensitivity of an affinity-based biosensor. Another object of the present invention is to provide a method of enhancing binding of a probe (P) and a target material in an affinity-based biosensor. Another object of the present invention is to provide an apparatus that can enhance binding of a probe and a target material.

A binding enhancing apparatus according to one embodiment of the present invention includes: a first electrode; a second electrode spaced from the first electrode; a channel unit electrically connected at a portion with the first electrode and electrically connected at another portion with the second electrode; a stimuli source electrically connected with the channel unit and applying an electric stimulus; and probes connected to the channel unit and complementarily bound with target materials to sense.

A method for binding enhancement according to one embodiment of the present invention includes: preparing a binding enhancing apparatus including at least one probe that is complementarily bound with a target material; positioning a solution containing target materials that are contemporarily bound with the probe onto the binding enhancing apparatus; and applying an electric stimulus by means of a stimuli source.

A biosensor according to one embodiment of the present invention includes: a first electrode; a second electrode spaced from the first electrode; a channel unit electrically connected at a portion with the first electrode and electrically connected at another portion with the second electrode; a stimuli source electrically connected with the channel unit and applying an electric stimulus; and probes connected to the channel unit and complementarily bound with target materials to sense.

A biosensor according to one embodiment of the present invention includes a semiconductor substrate, a gate on the substrate with an insulation film therebetween, a source and a drain on the semiconductor substrate, a probe on the gate, and a stimuli source electrically connected with the gate and applying an electric stimulus.

A biosensor array according to one embodiment of the present invention includes: a substrate; second electrodes covering a side of the substrate and having a plurality of holes exposing the substrate; a plurality of first electrodes disposed in the holes and spaced from the second electrode; a channel unit electrically connecting the second electrodes and the first electrode each other; probes connected to the channel unit; and a stimuli source applying an electric stimulus to any one of the first electrodes and any one of the second electrodes, in which the holes are arranged in an array.

A sensing method according to one embodiment of the present invention includes: preparing a biosensor including at least one probe that is complementarily bound with a target material; positioning a solution containing target materials that is complementarily bound with the probe onto the biosensor; applying an electric stimulus by means of a stimuli source; and sensing the target materials in a transient period of the applied electric stimulus.

According to the binding enhancing apparatus and the method for binding enhancementenhancement of an embodiment of the present invention, since the probe is bonded with the target material in a wider range while being extended and contracted by an electric stimulus from the stimuli source, the bonding ratio per unit time is improved. The target material can be effectively bonded with the probe, even if the mobility or the concentration of the target materials is low.

According to the biosensor and the sensing method of an embodiment of the present invention, since it is possible to perform sensing after the ions having opposite charge around the probe and the target materials are repelled away by an electric stimulus from the stimuli source, the sensing sensitivity of the target materials is improved. It is possible to effectively sense the target materials, even if the concentration of the target materials is low.

### BRIEF DESCRIPTION OF THE DRAWINGS

FIG. 1 is a cross-sectional view of a binding enhancing apparatus according to an embodiment of the present invention.

FIGS. 2A and 2B are cross-sectional views showing binding enhancing apparatuses formed in an array, according to an embodiment of the present invention.

FIG. 3 is a view showing a solution S containing a target material T on the binding enhancing apparatus according to an embodiment of the present invention.

FIG. 4 is a view showing that an electric stimulus has been applied by a stimuli source, in which the electric stimulus is negative charge.

FIG. 5 is a view showing that an electric stimulus has been applied by the stimuli source, in which the electric stimulus is positive charge.

FIG. 6 is a view showing an example of an electric stimulus applied by the stimuli source.

FIG. 7 is a flowchart illustrating an embodiment of a method of binding enhancement according to the present invention.

FIG. 8 is a cross-sectional view of a biosensor according to an embodiment of the present invention.

FIG. 9 is a view showing the types of electric stimuli having a step waveform applied by the stimuli source.

FIG. 10 is a schematic view separately showing the operation of the biosensor according to an embodiment of the present invention, with the lapse of time.

FIG. 11 is a view showing a transient period of an electric stimulus applied by the stimuli source.

FIG. 12 is a cross-sectional view schematically showing the biosensor according to the embodiment.

FIG. 13 is a view showing a solution containing target materials T on the biosensor according to the embodiment.

FIG. 14 is a flowchart illustrating an embodiment of a sensing method according to the present invention.

### DETAILED DESCRIPTION

The description of the one embodiment of present invention is just examples for structural and functional illustration, and thus the scope of the present invention should not be construed as being limited by these examples. That is, since the present invention may be variously modified and have several embodiments, the scope of the present invention should be understood as including equivalents by which the spirit of the present invention can be achieved.

The terms used herein should be understood as follows.

The terms, such as “first”, “second” etc, are for discriminating one component from another component, but the scope is not limited to the terms. For example, the first component may be named the second component and the second component may also be similarly named the first component.

It is to be understood that when one element is referred to as being “on” or “above” another element, it may be directly on another element but other elements may be disposed between them. On the other hand, it is to be understood that when one element is referred to as being “in contact with” another element, it may be connected to another element without other elements therebetween. Further, other expressions describing the relationships of components, that is, “interposed” and “directly interposed, “between” and “directly between”, or “close to” and “directly close to” should be understood in the same way.

Singular forms are intended to include plural forms unless the context clearly indicates otherwise. It will be further understood that the terms “comprises” or “have” as used in this specification specify the presence of stated features, steps, operations, components, parts, or a combination thereof, but do not preclude the presence or addition of one or more other features, numerals, steps, operations, components, parts, or a combination thereof.

The steps may be generated in different ways from the order described herein unless the context clearly indicates the order otherwise. That is, the steps may be generated in the order described or simultaneously, but they may be performed in reverse direction.

In the drawings referred to herein for describing embodiments of the present invention, the size, height, and thickness etc. may be intentionally exaggerated and not enlarged or reduced in accordance with scales. Further, some components may be intentionally reduced and some other components may be intentionally enlarged.

Unless defined otherwise, it is to be understood that all the terms used herein including technical and scientific terms have the same meaning as those as understood by those who are skilled in the art. It should be understood that the terms defined by dictionaries must be identical with the meanings within the context of the related art, and they should not be ideally or excessively formally defined unless the context clearly dictate otherwise.

A binding enhancing apparatus and a biosensor according to an embodiment of the present invention may have the same or similar external appearance and configuration. Accordingly, it should be understood that the biosensor may be described with reference to the drawings related to the binding enhancing apparatus of the present invention.

### Embodiment 1

An embodiment of a binding enhancing apparatus according to the present invention is described hereafter with reference to the accompanying drawings. A binding enhancing apparatus according to an embodiment of the present invention includes a first electrode, a second electrode spaced from the first electrode, a channel unit electrically connected at a portion with the first electrode and electrically connected at another portion with the second electrode, a stimuli source electrically connected with the channel unit and applying an electric stimulus, and probes P connected to the channel unit and complementarily bound with target materials to sense.

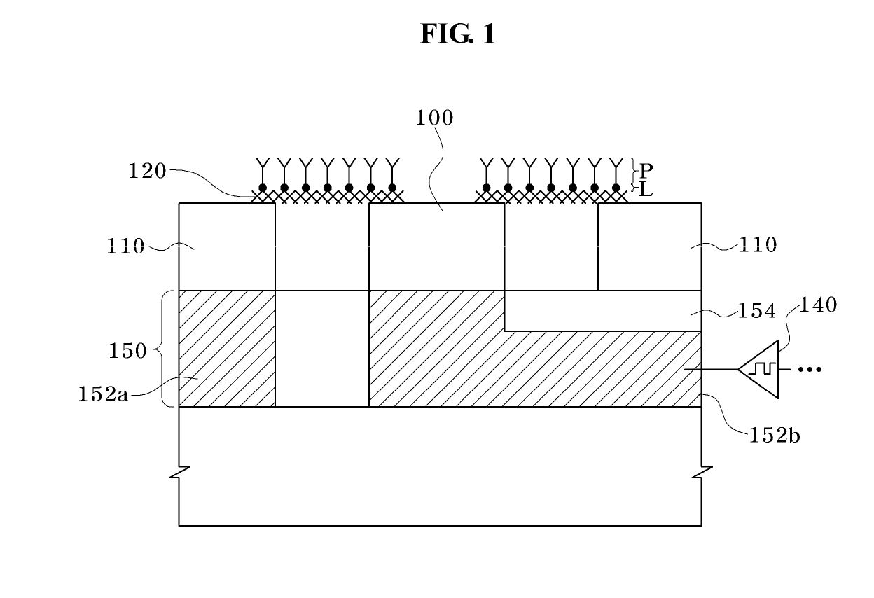

FIGS. 1 to 7 are views illustrating a binding enhancing apparatus and a method of binding enhancement according to an embodiment of the present invention. FIG. 1 is a cross-sectional view of a binding enhancing apparatus according to an embodiment of the present invention. Referring to FIG. 1, in an embodiment, a first electrode 100 and a second electrode 110 are made of conductive metal. For example, the first electrode 100 and the second electrode 110 contain gold (Au). As shown in the figure, the first electrode 100 and the second electrode 110 are spaced from each other. In an example, an air gap is disposed in the gap between the first electrode 100 and the second electrode 110. In another example, the gap between the first electrode 100 and the second electrode 110 may be filled with an insulating film such as an oxide film and a nitride film.

A channel unit 120 electrically connects the first electrode 100 and the second electrode 110 each other. That is, a portion of the channel unit 120 is electrically connected with the first electrode 100 and another portion is electrically connected with the second electrode 110. For example, the channel unit is a single semiconductor material. As another example, the channel unit is formed by electrically connects at least one of a plurality of semiconductor materials, nano tubes, nano wires, nano rods, nano ribbons, nano films, and nano balls each other. As described above, at least one channel sub-unit electrically connected with the first electrode is electrically connected with at least another one channel sub-unit and they are electrically connected with at least one channel sub-unit electrically connected with the second electrode.

A stimuli source 140 is electrically connected and applies an electric stimulus to the channel unit 120. As an embodiment, the stimuli source 140 applies a pulse, which may be, for example, a step pulse, a rectangular pulse, a triangular pulse, and a sinusoidal pulse, and a pulse formed by linear superposition of the pulses or any one or more of the pulses may be applied in time sequence. As an embodiment, the stimuli source 140 may be electrically connected with the first electrode 100 and apply an electric stimulus to the channel unit 120. When the stimuli source keeps applying a direct current or an electric stimulus with a low frequency, an unexpected reaction such as electrolysis of a solution may be generated or the probe P may get denatured at the first electrode electrically connected with the stimuli source. Accordingly, the voltage, the frequency, or the duration period of the electric stimulus that is applied by the stimuli source is controlled to prevent an unexpected reaction or denaturation of the probe P. In an example, it is possible to swing the applied electric stimulus in the range of −10V to 10V. In another example, the applied electric stimulus may have a frequency of 10 GHz, more than zero.

The probe P is immobilized to the channel unit 120 and complementarily bound with a target material. In an embodiment, when the target material is DNA (DeoxyriboNucleic Acid) having a specific base sequence, the probe P is a material having a sequence that is complementarily bound with the base sequence of the target material. Similarly, in order to detect DNA, RNA (RiboNucleic Acid), protein, hormone, and antigen etc., a material that is complementarily bound with the DNA, RNA, protein, hormone, and antigen etc. is used for the probe P. Though described below, because the probe P should be deformed by an electric stimulus that is applied by the stimuli source, it should have specific charge. However, whether the charge is positive charge or negative charge does not matter and whether the amount of charge is large or small also does not matter, as long as it has charge. Although the probe P may be directly connected with a channel unit, it may be connected to the channel 120 by a linker L, as in the embodiment shown in the drawings. For example, the linker is a nano particle containing metal such as gold and silver. For example, the linker L may have a double structure of a first linker and a second linker. That is, the second linker may be a nano particle containing metal such as gold and silver and the first linker may be a linker for connecting the probe to the the second linker. In one embodiment, the first linker could also have a double structure that may include an organic compound region and a thiol group treated region. The organic compound region contains an amine group or a carboxyl group for bonding with the probe and the region treated with a thiol group is for bonding with the nano particle, which is the second linker.

In another embodiment, the double structure of the first linker may include a region having an antigen or an antibody, depending on the probe.

In another embodiment, a probe may be connected to a channel without a linker. For example, when DNA is used as a probe, one end of the DNA may be treated with a thiol group and directly connected to a channel unit.

In an embodiment, a wire layer 150 includes conduction patterns 152a and 152b and an insulation pattern 154 and is disposed under the layer on which the first electrode 100 and the second electrode 110 are. The conduction pattern 152a electrically connects the second electrode 110 and a second electrode each other. The conduction pattern 152b electrically connects the first electrode 100 and the stimuli source 140 each other. The insulation pattern 154 insulates the elements requiring electric insulation, such as in between the conduction pattern 152a and the conduction pattern 152b or the conduction pattern and the first electrode or the second electrode. In an example, the conduction patterns 152a and 152b are made of a conductive material, that is, a high conductive material such as gold, silver, copper, and aluminum. In an example, the insulation pattern is an insulation film, for example, an oxide film and a nitride film.

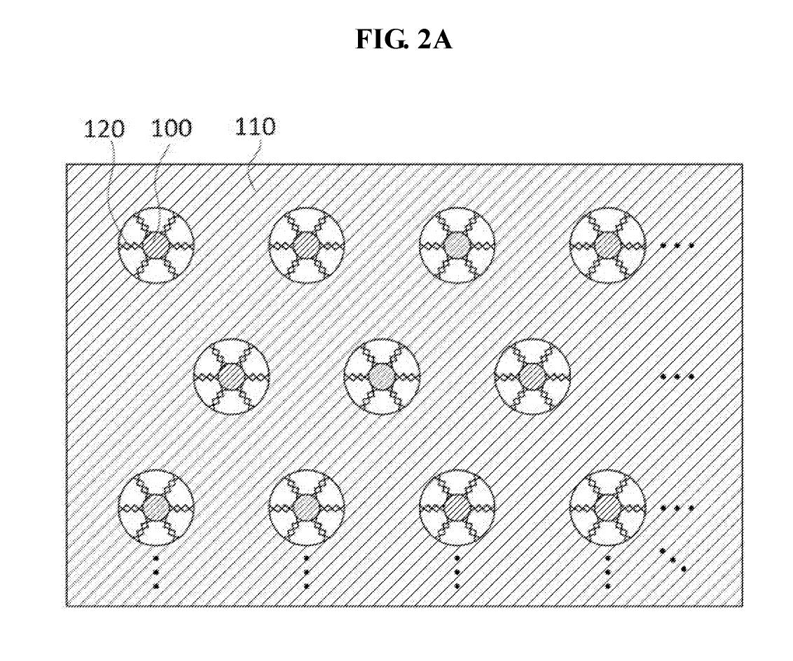

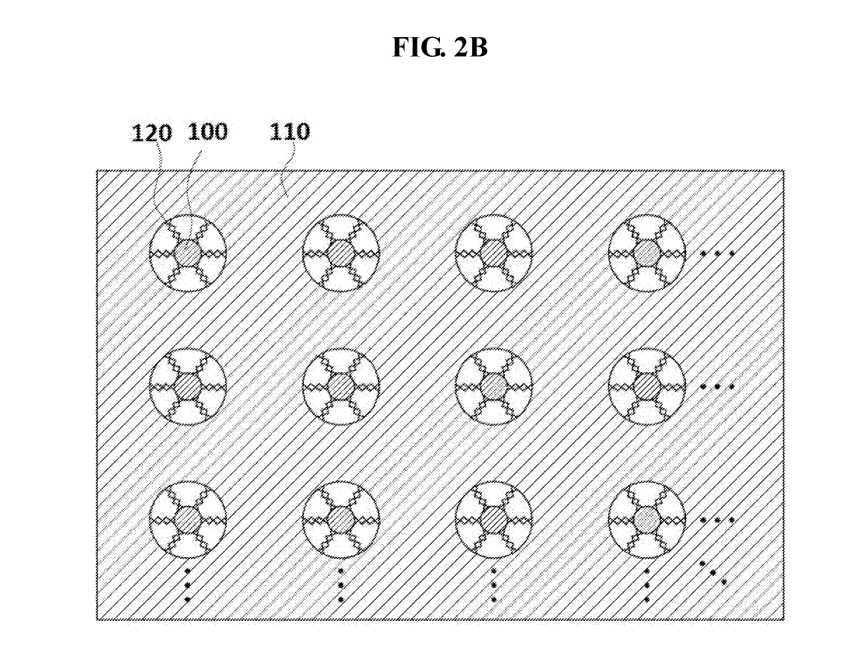

FIGS. 2A and 2B are cross-sectional views showing binding enhancing apparatuses formed in an array, according to an embodiment of the present invention. FIG. 2A exemplifies that binding enhancing apparatuses according to an embodiment of the present invention are disposed in an array, and binding enhancing apparatuses in one line and binding enhancing apparatuses in another line may be alternately disposed in an array, as in FIG. 2B. Further, the array may be changed in other ways not shown in the figures and the number of the sensors arranged transversely and longitudinally may be changed in accordance with embodiments.

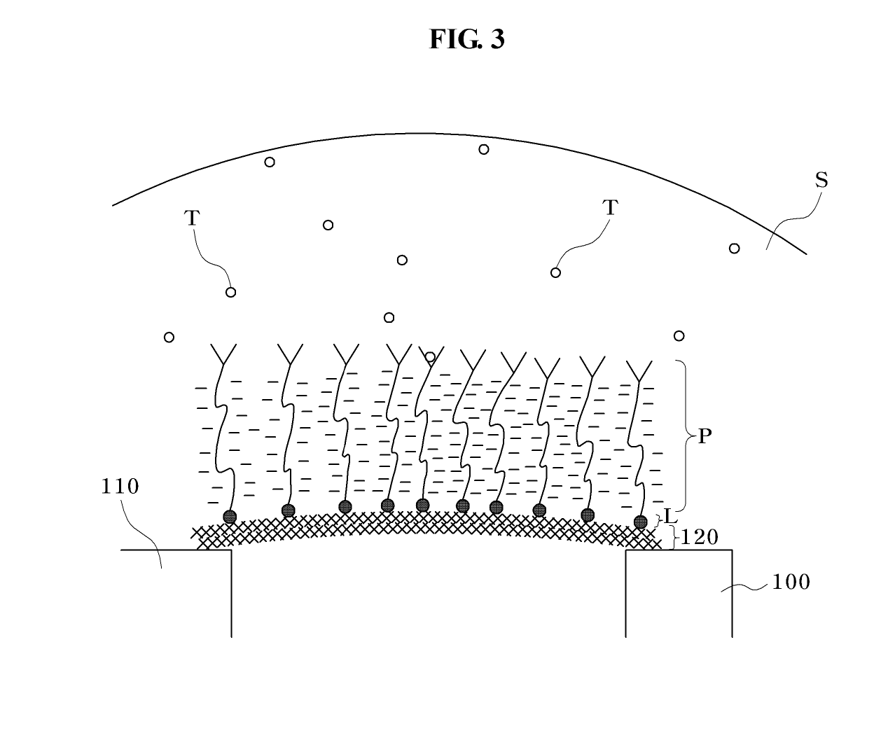

FIG. 3 is a view showing a solution S containing target materials T on the binding enhancing apparatus according to an embodiment of the present invention. A binding enhancing apparatus and a method of binding enhancement according to an embodiment of the present invention are described with reference to FIGS. 3 to 7. Referring to FIG. 3, the probes P are in a relaxed state without extending or contracting, as shown in the figure, before the stimuli source applies a stimulus. Further, the probes P have charge, as described above. Although it is exemplified in the embodiment that the probes P have negative charge, the probes P may have any of positive charge and negative charge, as described above. When the solution S containing the target materials T is positioned on the binding enhancing apparatus, the probes P and the target materials are bound by diffusion of the target materials. Accordingly, the binding rate per unit time between the target material and the probe P depends on the diffusion speed of the target material, and when the concentration of the target materials in the solution is low or the diffusion speed of the target material is low, the binding rate per unit time between the target material and the probe P decreases.

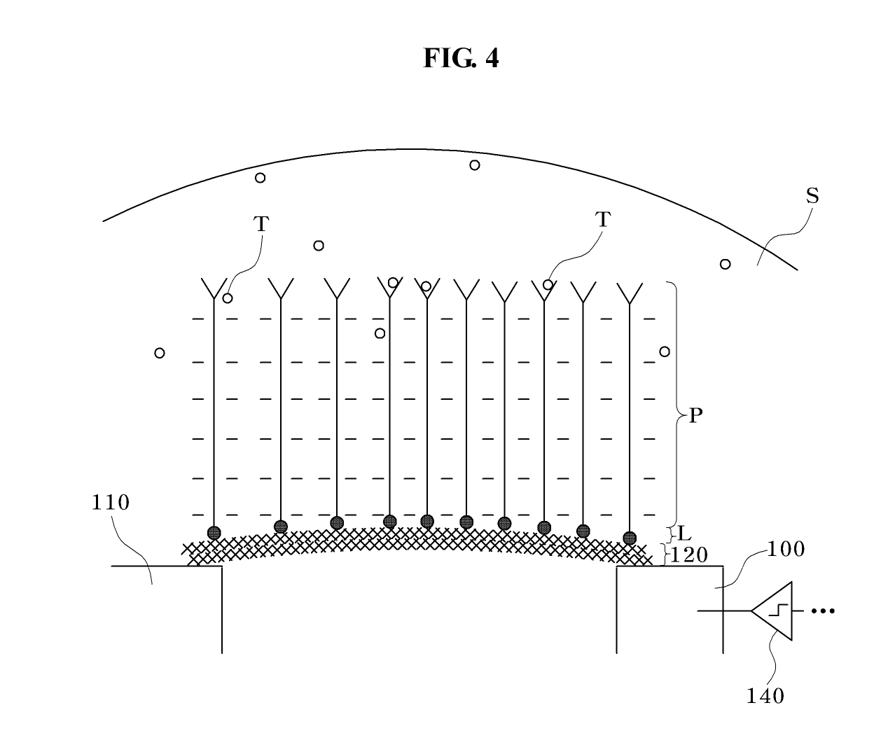

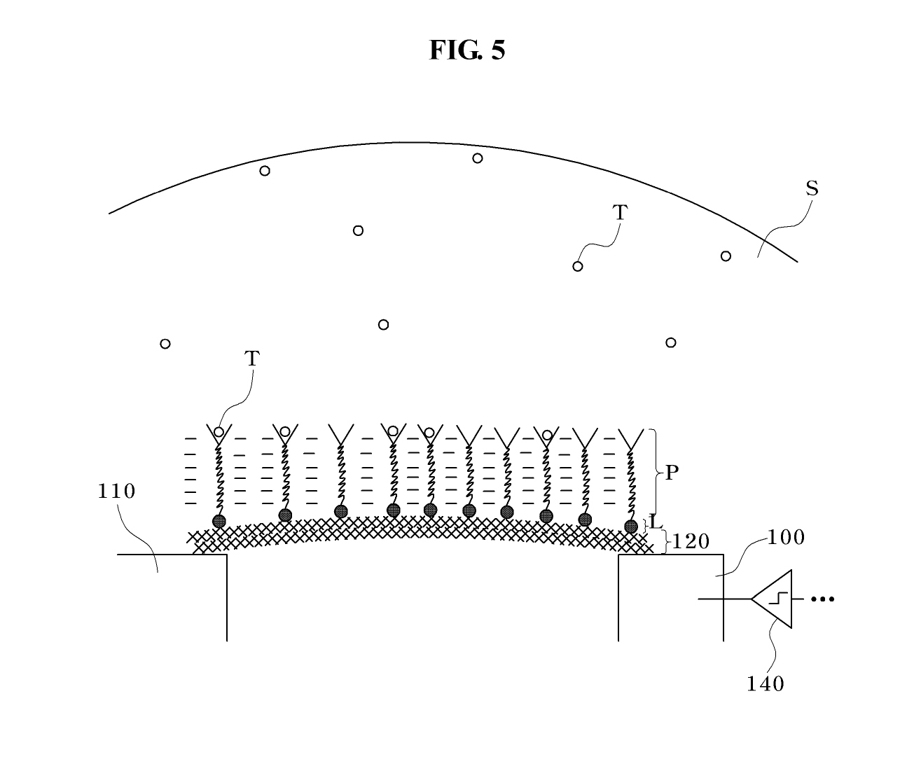

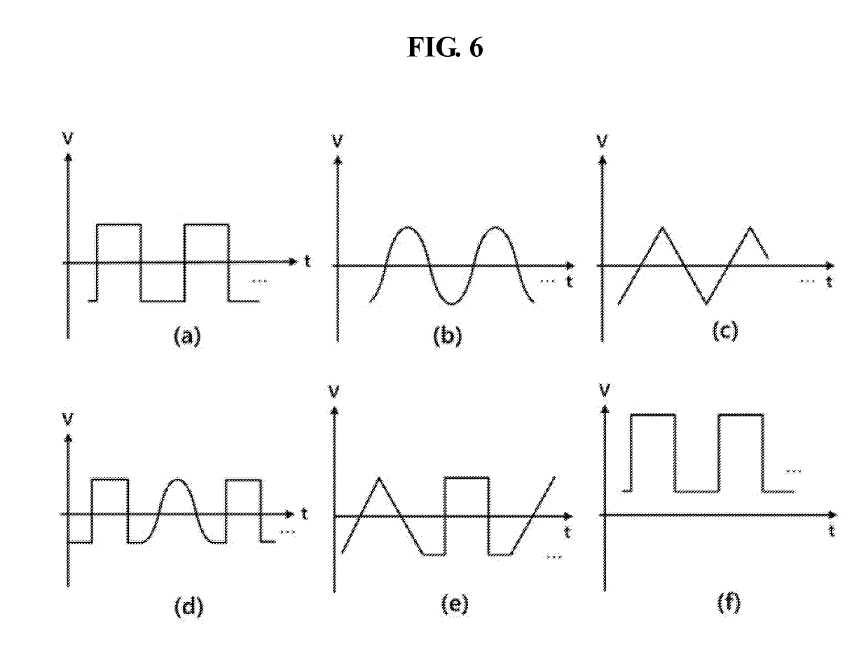

FIG. 4 is a view showing that an electric stimulus has been applied by a stimuli source, in which the electric stimulus is negative charge and FIG. 5 is a view showing that an electric stimulus has been applied by the stimuli source, in which the electric stimulus is positive charge. FIG. 6 is a view showing an example of an electric stimulus applied by the stimuli source. Referring to FIGS. 4, 5, and 6, the stimuli source 140 applies an electric stimulus to the probe P through the first electrode 100. For example, although it is shown in FIGS. 4 and 5 that the electric stimulus applied by the stimuli source 140 is a rectangular pulse, it is just an example, and the electric pulse that is applied by the stimuli source 140 may be a rectangular pulse, as shown in FIG. 6a, a sinusoidal pulse, as shown in FIG. 6b, and a triangular pulse, as shown in FIG. 6. Further, it has only to be an electric pulse that changes in amplitude or polarity with the lapse of time, such as a pulse having alternate rectangular pulses and sinusoidal pulses in time sequence or a pulse having alternate triangular pulses and rectangular pulses in time sequence. Further, as shown in FIG. 6f, a signal with predetermined offset and a signal with overlapped pulses may be applied. However, although it is shown in FIG. 6 that all of the pulses have the same swing, it is just an example, and the swing of the pulses may change with the lapse of time and pulses having different swing may alternate in time sequence. Further, it may be possible to apply any one pulse during a predetermined period, not apply any stimulus during a suspension period, and then apply again an electric stimulus.

Referring to FIGS. 4 and 5, the probe P is deformed by an electric stimulus applied by the stimuli source 140. As an embodiment, the stimuli source 140 applies an electric stimulus alternately having positive voltage and negative voltage at predetermined periods. Since negative voltage is applied to the channel unit electrically connected with the stimuli source 140 in the section where the pulse applied by the stimuli source 140 has negative voltage, the probe P having negative charge repels and extends, as shown in FIG. 4. Positive voltage is applied to the channel unit 120 connected with the stimuli source in the section where the pulse applied by the stimuli source has positive voltage. Accordingly, the probe P having negative charge contracts toward the channel unit 120, as shown in FIG. 5. Accordingly, the probe P repeats extending and contracting in accordance with the pulse applied with alternate polarities since the target materials T contained in the solution are freely moved and uniformly distributed throughout the solution, not only the target materials in the solution are bound with the probes P by diffusing to the probes, but the probes are bound with the target materials over a wider range by repeating extending and contracting, such that the binding ratio per unit time is improved.

As another embodiment, when the target material, the solution containing the target material, or the probe P is vulnerable to an applied electric stimulus and an electric pulse is continuously applied, the target material, the solution containing the target material, or the probe P may be denatured. In this case, it is possible to apply an electric stimulus such that the target material, the solution containing the target material, and the probe P are not denatured, by alternating a section where a pulse is applied and a suspension period where a pulse is not applied, at predetermined periods. As an example, when the electric stimulus applied by the stimuli source has negative voltage, the probe P extends in the section where negative charge is applied, as described above. Next, since the stimuli source does not apply a voltage in the suspension period, the probe P keeps in the relaxation state without extending or contracting. Therefore, the probe P is bound with the target material in the solution through the extension and relaxation states, when negative voltage and a suspension period are alternately applied. Similarly, in the embodiment, since the probe P is bound with the target material while extending or contracting, the binding ratio per unit time is improved. Although it is exemplified in the embodiment that the section where negative voltage is applied and the suspension period are alternated, the section where positive voltage is applied and the suspension period may be alternated, depending on the polarity of the charge of the probe P. In another embodiment, it is possible to apply a pulse having the positive maximum and the positive minimum by superpositioning a direct current and a pulse, as shown in FIG. 6f, by means of the stimuli source.

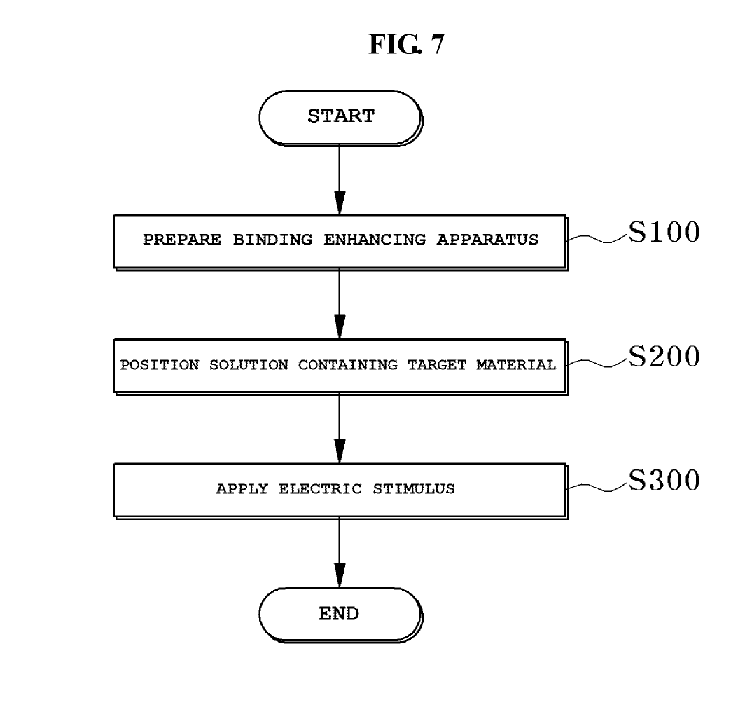

An embodiment of a method for binding enhancement according to the present invention is described hereafter with reference to the accompanying drawings. FIG. 7 is a flowchart illustrating an embodiment of a method of binding enhancement according to the present invention. The method for binding enhancement according to an embodiment of the present invention includes: preparing a binding enhancing apparatus including at least one probe that is complementarily bound with a target material (S100); positioning a solution containing target materials onto the binding enhancing apparatus (S200); and applying an electric stimulus by means of a stimuli source (S300). The part overlapping the binding enhancing apparatus according to an embodiment of the present invention may not be described to make the description brief and clear.

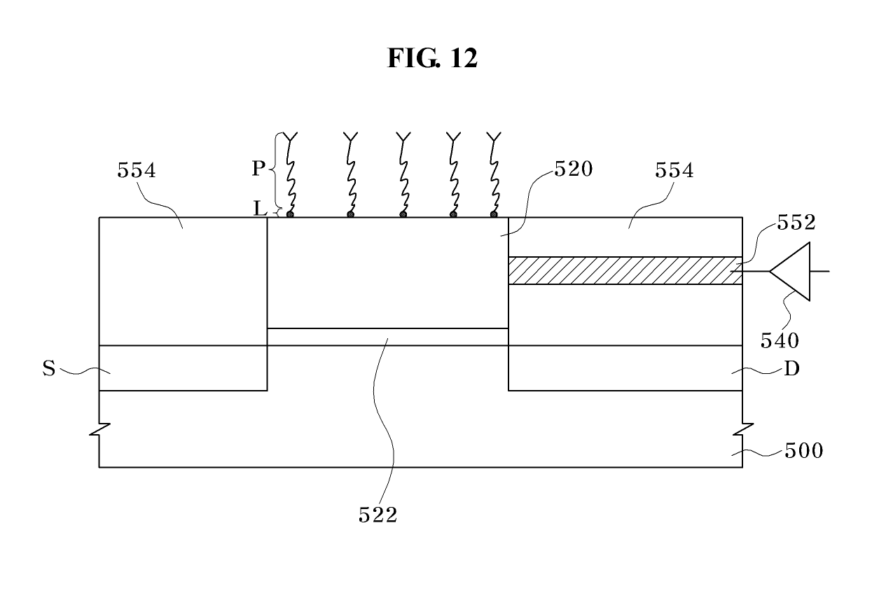

Referring to FIG. 7, a binding enhancing apparatus including a probe P that is complementarily bound with a target material is prepared (S100). As an embodiment, the binding enhancing apparatus may include the probes P electrically connecting the first electrode 100 and the second electrode 110 each other, as shown in FIG. 1. As another embodiment, the binding enhancing apparatus may be formed in an array, as shown in FIGS. 2A and 2B, and may be formed in any types of arrays, which are not shown in the figures. As another embodiment, the binding enhancing apparatus may include probes on gate surface, as shown in FIG. 12.

A solution containing target materials is positioned onto the binding enhancing apparatus (S200). The material of the probe P in the binding enhancing apparatus may predetermined charge, the probe P is in a relaxation state, when an electric stimulus is not applied by a stimuli source, and the target materials T are uniformly distributed in the solution. Accordingly, the binding ratio per unit time of the target material and the relaxed probe P depends on the diffusion speed of the target material. In particular, when the mobility or the concentration of the target materials is low, the binding ratio of the probe P and the target material further decreases.

A stimuli source applies an electric stimulus (S300). In an embodiment, the electric stimulus applied by the stimuli source may be at least one of a rectangular pulse, a triangular pulse, a sinusoidal pulse, and a linear superposition or combination of them in time sequence, as shown in FIG. 6. In another embodiment, though not shown in the figures, the stimuli source may apply an electric stimulus while alternating a section where an electric stimulus is applied and a predetermined suspension period.

In this step, the probe P contracts or extends, as shown in FIG. 4 or 5, depending on the electric stimulus applied by the stimuli source. Therefore, the probe P can be bound with the target material over a wider range, such that the binding ratio and binding number per unit time increase. In particular, the target material can be effectively bound with the probe P, even though the mobility or the concentration of the target materials is low.

### Embodiment 2

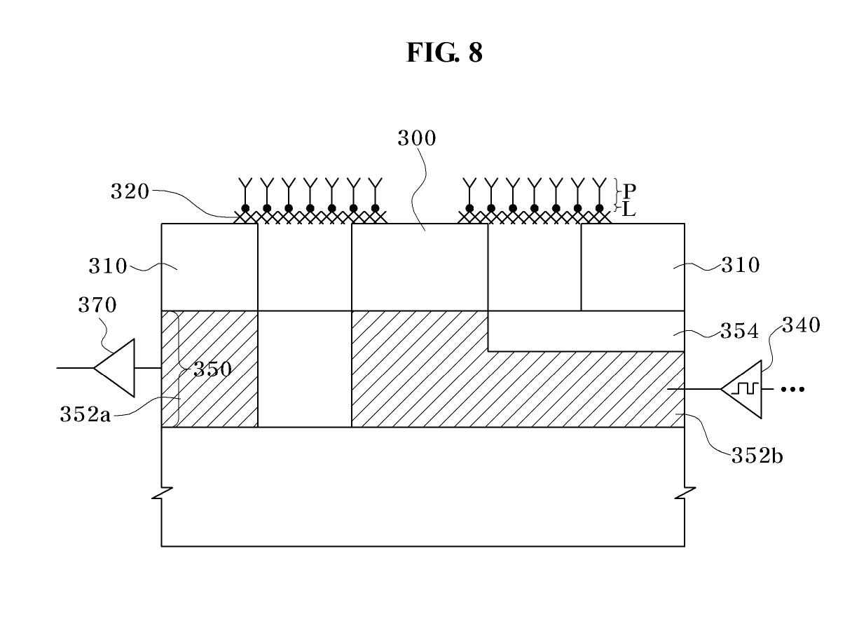

A biosensor according to an embodiment of the present invention is described hereafter with reference to the accompanying drawings. The part overlapping the binding enhancing apparatus according to an exemplary embodiment of the present invention may not be described to make the description brief and clear. FIGS. 8 to 13 are views illustrating a biosensor according to an embodiment of the present invention. FIG. 8 is a cross-sectional view of a biosensor according to an embodiment of the present invention. Referring to FIG. 8, a biosensor according to an embodiment of the present invention includes a first electrode 300, a second electrode 310 spaced from the first electrode 300, a channel unit 320 electrically connected at a portion with the first electrode and electrically connected at another portion with the second electrode, a stimuli source 340 electrically connected with the channel unit and applying an electric stimulus, and probes P connected to the channel unit and complementarily bound with target materials to sense. Although the probe P may be directly connected with a channel unit, it may be connected to the channel 120 by a linker L, as in the embodiment shown in the drawings. For example, the linker is a nano particle containing metal such as gold and silver. For example, the linker L may have a double structure of a first linker and a second linker. That is, the second linker may be a nano particle containing metal such as gold and silver and the first linker may be a linker for connecting the probe to the the second linker. In one embodiment, the first linker could also have a double structure that may include an organic compound region and a thiol group treated region. The organic compound region contains an amine group or a carboxyl group for bonding with the probe and the region treated with a thiol group is for bonding with the nano particle, which is the second linker.

In another embodiment, the double structure of the first linker may include a region having an antigen or an antibody, depending on the probe.

In another embodiment, a probe may be connected to a channel without a linker. For example, when DNA is used as a probe, one end of the DNA may be treated with a thiol group and directly connected to a channel unit.

In an embodiment, the probe P is a material that is complementarily bound with a target material to sense. For example, when the target material is one of DNA, RNA, enzyme, protein, and hormone, the probe P may be any one of DNA, RNA, enzyme, protein, and hormone that is complementarily bound with the target material.

In an embodiment the biosensor according to the present invention may have an array shape. FIGS. 2A and 2B are cross-sectional views showing biosensors formed in an array, according to an exemplary embodiment of the present invention. Referring to FIGS. 2A and 2B, a biosensor according to the one embodiment of present invention includes a substrate, second electrodes covering a side of the substrate and having a plurality of holes exposing the substrate, a plurality of first electrodes formed in the holes and spaced from the second electrode, a channel unit electrically connecting the second electrodes and the first electrode each other, probes P connected to the channel unit, and a stimuli source applying an electric stimulus to any one of the first electrodes and any one of the second electrodes, in which the holes may be arranged in an array.

FIG. 2A exemplifies that biosensors according to an exemplary embodiment of the present invention are disposed in a rectangular array, and sensors in one line and sensors in another line may be alternately disposed in an array, as in FIG. 2B. Further, the array may be changed in other ways not shown in the figures and the number of the sensors arranged transversely and longitudinally may be changed in accordance with embodiments.

When the biosensors according to an embodiment of the present invention are disposed in an array, the contact area between the second electrode and a solution containing a target material increases, such that capacitance between the second electrode and the solution also increases. Accordingly, the potential of the solution does not change, even if an electric stimulus is applied through the stimuli source electrically connected with the first electrode. As the sensors according to the one embodiment of present invention are disposed in an array, as described above, it is not required to provide an additional electrode that applies a predetermined voltage to the solution containing target materials in order to keep the potential of the solution.

In an exemplary embodiment, a wire layer 350 includes conduction patterns 352a and 352b and an insulation pattern 354 and is disposed under the layer on which the first electrode 300 and the second electrode 310 are. The conduction pattern 352a electrically connects the first electrode with a first electrode or the first electrode 300 with a read-out circuit unit 370. The conduction pattern 152b electrically connects the second electrode 310 and the stimuli source 340 each other. The insulation pattern 354 insulates the elements requiring electric insulation, such as in between the conduction pattern 352a and the conduction pattern 352b or the conduction pattern and the first electrode or the second electrode. In an example, the conduction patterns 352a and 352b are made of a conductive material, that is, a high conductive material such as gold, silver, copper, and aluminum. In an example, the insulation pattern is an insulation film, for example, an oxide film and a nitride film.

The read-out circuit unit 370 senses and outputs charge induced to the channel unit by the target material bound to the probe P. In an embodiment, the read-out circuit includes a charge amplifier. The read-out circuit unit 370 stores the charge induced to the channel unit into a capacitor (not shown) connected to an input terminal of the charge amplifier and then the charge amplifier outputs an electric signal proportional to the amount of charge stored in the capacitor. However, the configuration of sensing the charge induced to the channel unit by the target material and then outputting an electric signal is apparent to those skilled in the art and may be implemented in other ways than the charge amplifiers described above.

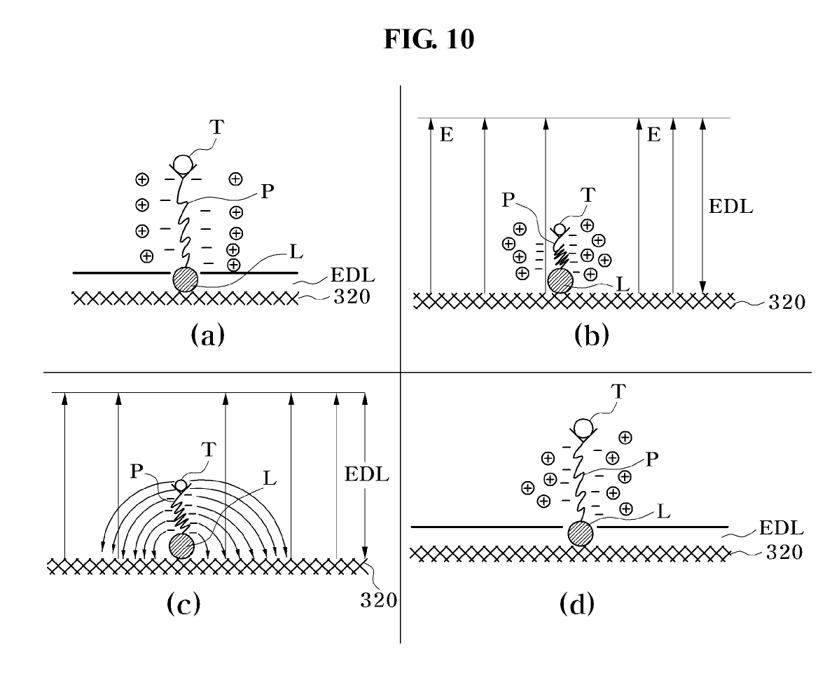

The operation of the biosensor according to an embodiment of the present invention is described with reference to the accompanying drawings. FIG. 10 is a schematic view separately showing the operation of the biosensor according to an embodiment of the present invention, with the lapse of time, and which schematically showing the operation from a steady state before the stimuli source applies an electric stimulus of a step waveform to a steady state after the stimuli source applies an electric stimulus. FIG. 10a is a view showing the steady state right before a step waveform is applied.

FIG. 10a shows a probe P and a target material T bound to each other. When a solution containing target materials T is positioned on the sensor, the target material diffuses and comes in contact with the probe P, such that the binding ratio per unit time is low. Further, when the mobility and the concentration of the target material T are low, the binding ratio per unit time between the probe P and the target material further decreases. When a solution containing target materials is positioned on the first electrode and the second electrode and a stimulus is applied through a stimuli source by performing the method of binding enhancenhancement according to an embodiment of the present invention, the binding ratio and binding number per unit time between the probe P and the target material increase, which was described above. Further, although FIG. 10 shows only one probe P, this is for making the description brief and easy, and it is apparent that a target material is actually bound to a plurality of probes P.

When the target material T is complementarily bound to the probe P, charge is induced to the channel unit 320 by the charge of the target material. That is, when the target material has negative charge, positive charge is induced to the channel unit by the negative charge, or when the target material has positive charge, negative charge is induced to the channel unit by the positive charge. The concentration etc. of the target material can be measured by sensing the charge, which is induced, as described above, with a read-out circuit 570.

However, when the target material T is in an electrolyte solution such as blood and sensing is performed in this way, sensing sensitivity decreases. As shown in FIG. 10a, the probe P and the target material T bound to the probe P have charge and ions having opposite charge in an electrolyte solution and ions having charge opposite to the ions having the opposite charge (that is, having the same charge type as that of the probe material) surround the target material, such that opposite charge is not induced to the channel unit by the target material. This effect is called a charge screening effect. That is, charge is induced to the channel unit when electric field flux from the target material reaches the channel unit, but most of the electric field flux from the target materials are blocked by the ions having opposite charges around the target materials. Therefore, the amount of charge that is induced to the channel unit decreases by the charge screening effect and the sensitivity of the affinity-based biosensor also decreases.

FIG. 10b is a schematic view showing that status at t=0+, right after a stimulus source applies a step waveform. Referring to FIG. 10, an electric stimulus having a step waveform applied by a stimuli source is applied to the channel unit through the conduction pattern 352b and the first electrode 300. After the electric stimulus is applied, the probe P (330) is contracted and an EDL (Electrical Double Layer) is expanded by the applied positive voltage. That is, the positive ions and the negative ions in the EDL are redistributed by an electric field due to the electric stimulus from the stimuli source and the regular double layer arrangement of the positive ions and the negative ions is broken during the redistribution, and accordingly, the positive ions and the negative ions are randomly mixed and expand to the range that the electric field reaches. Therefore, the electric field penetrates into the expanding region of the EDL without being shielded by the EDL, such that it influences the probe P and the positive ions around the probe P.

Referring to FIG. 10c, the electric field penetrates into the EDL while the EDL expands in a transient period after t=0+, such that the positive ions around the probe P and the target material are repelled away. Accordingly, the electric field flux from the target material travel to the channel unit 530 and induce charge to the channel unit without being blocked by the surrounding positive ions in the transient period, such that the read-out circuit unit 370 can measure the concentration of the target materials in the solution by sensing the induced charge.

Referring to FIG. 10d, the EDL that has expanded is redistributed back to the surface of the channel unit and shields the electric field in the steady state after the transient period at t>0. Accordingly, since the electric field cannot penetrate into the EDL, even if the stimuli source 340 keeps applying positive voltage, the positive ions that have been repelled away are attracted by the negative charge of the probe P and the target material and surround again the probe P. Therefore, when the target material is measured in the steady state after the transient period is ended, the sensing sensitivity is lower than that when it is measures in the transient period.

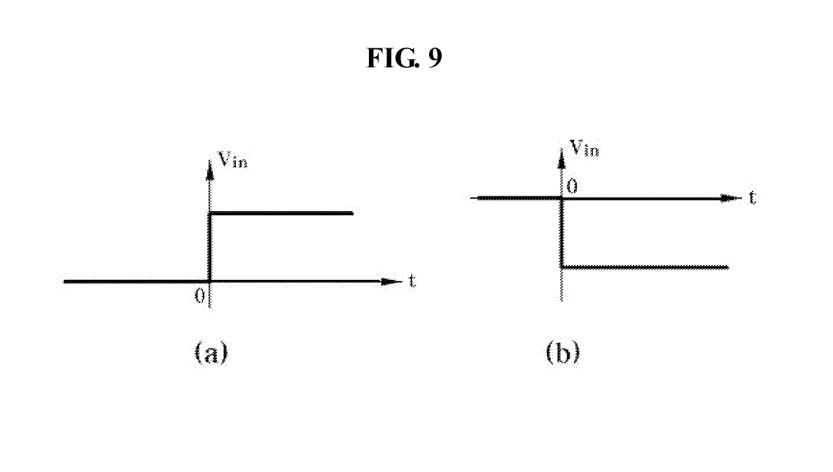

In the embodiment, it is exemplified that the probe P and the target material T have negative charge and the stimuli source applies a step waveform having positive potential shown in FIG. 9a, the probe P and the target material T may have negative charge and the stimuli source may apply a step waveform having negative potential shown in FIG. 9b. Even though negative potential is applied, the EDL expands and the electric field penetrates into the expansion region of the EDL, such that the ions close to the probe P and the target material T can be repelled away from the probe P and the target material T. Accordingly, it is possible to measure the charge induced to the channel unit in the transient period until the EDL is redistributed after the ions are repelled away from the probe P and the target material T.

Further, when the probe P and the target material T have positive charge, the stimuli source may apply a step waveform having negative potential, as shown in FIG. 9b. Further, after any one of the pulses shown in FIG. 6 is applied, sensing can be performed in the corresponding transient period. However, this is apparent to those skilled in the art and is not described for brief and clear description.

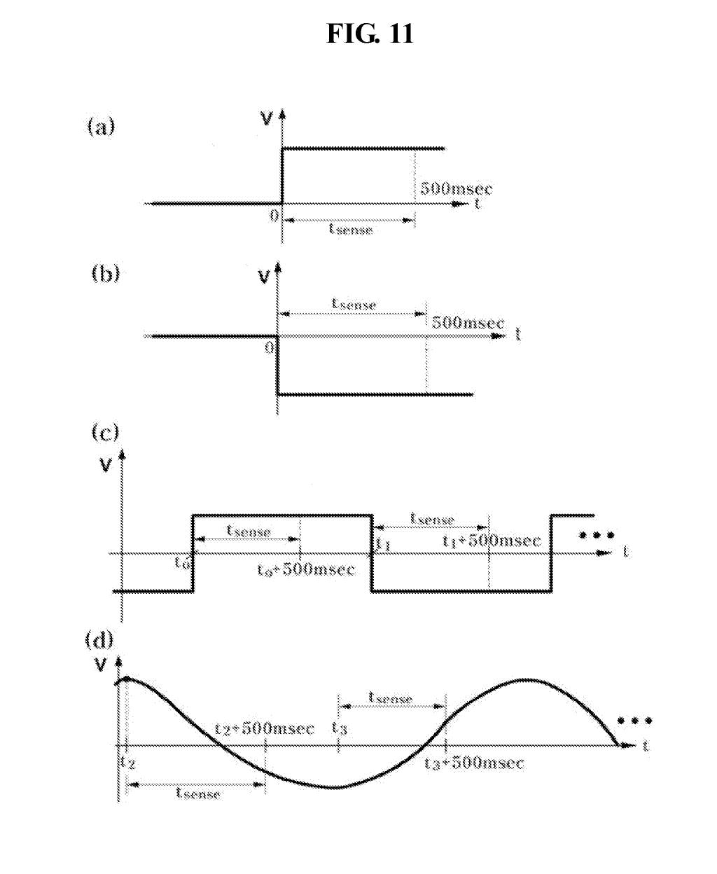

FIG. 11 is a view showing a transient period of an electric stimulus applied by the stimuli source. Referring to FIG. 11, an electric stimulus is applied and then sensing is performed in the transient period. The transient period means a state until an EDL expands after a stimulus is applied, as described above, the ions around the probe P and the target T are moved by the penetrating electric field, and the ions surround again the probe P and the target material T by the EDL redistributed and shielding the electric field. Accordingly, the duration time of the transient period depends on the structure of the biosensor and the concentration of the ions around the probe P and the target material T too. That is, the transient period decreases, when the concentration of the ions is high, whereas the transient period increases, when the concentration of the ions is low.

In general, for a buffer solution containing Sodium chloride (NaCl) use for sensing of a biomaterial, the period from the point of time of applying potential to O(10̂(−6)) sec where an EDL is redistributed can be considered as the transient period, and to 0−O(10̂(−2)) sec when it is measured in water with impurities refined at a predetermined degree. Further, when other ions are refined at higher degree of refining on a predetermine measuring platform than the state described above, the transient period may increase up to 500 msec, and the transient period may decrease up to 0.1 nsec, for a buffer solution containing NaCl with high concentration.

As an embodiment, PBS (Phosphate Buffered Saline) or TE (Tris-EDTA) buffer solution may be used for sensing a biomaterial. When ions are contained with high concentration in the PBS or TE buffer solution, the transient period may be about 0.1 nsec when a target material is sensed, whereas the transient period may be about 500 msec when the ions are contained with low concentration.

For example, referring to FIG. 11a, when a stimuli source applies a stimulus having a step waveform of positive potential, sensing is performed from the rising edge up to a maximum of 500 msec (tsense). As another example, referring to FIG. 11b, when a stimuli source applies a stimulus having a step waveform of negative potential, sensing is performed from the falling edge up to a maximum of 500 msec (tsense). As another example, referring to FIG. 11c, when a stimuli source applies a stimulus having a rectangular pulse waveform, sensing is performed in the section from the rising edge t0 to 500 mse (tsense) and/or sensing is performed in the range from the falling edge t1 to 500 msec (tsense). As another example, referring to FIG. 11d, when a stimuli source applies an electric stimulus having a sine waveform, sensing is performed in the section from the end of the rising edge t2 to 500 mse (tsense) and/or sensing is performed in the range from the end of the falling edge t3 to 500 msec (tsense). However, the figures shows the sections where sensing is performed, sensing may keep performed for the sensing section (tsense), and the sensing may be performed one time or more in the corresponding sections. Further, when a continuous pulse is applied, as in FIGS. 11c and 11d, sensing may be performed in the section from the rising edge and/or the falling edge to 500 msec.

However, sensing for 500 msec shown in the figures means sensing within 500 msec after a stimulus is applied, which does not means that 500 msec is necessary or sensing should be performed for 500 msec, and sensing may be performed in any one section for 500 msec after a stimulus is applied.

Accordingly, the section where a stimulus is applied and sensing is performed depends on the concentration of the ions in the solution or the concentration of the target material, such that the transient period may be about 0.1 nsec, in which the frequency of the applied stimulus may be a maximum of 10 GHz. Further, the transient period may be 500 msec, in which the frequency of the applied stimulus may be a maximum of 2 Hz.

Another embodiment of the biosensor according to the present invention is described. A biosensor according to the embodiment includes a semiconductor substrate, a gate on the substrate with an insulation film therebetween, a source and a drain on the semiconductor substrate, at least one probe P on the gate, and a stimuli source electrically connected with the gate and applying an electric stimulus. In an embodiment the biosensor according to the embodiment further includes a read-out circuit unit electrically connected with the source S or the drain D. The configuration overlapping those of the embodiments described above is not described to describe briefly and clearly the embodiment.

FIG. 12 is a cross-sectional view schematically showing the biosensor according to the embodiment. Referring to FIG. 12, a source S and a drain D are on a semicircular substrate 500. In an embodiment, though not shown, a read-out circuit unit that reads out and processes a signal outputted from a biosensor, according to the embodiment, is connected to the source S or the drain D. A gate 502 is on the substrate with an insulation film 522 therebetween and a probe P that is complementarily bound with a target material T to sense using the biosensor according to the embodiment is on the gate. For example, the probe P may be disposed on the gate through a linker L. In an embodiment, a conduction pattern 522 electrically connects the gate 520 and a stimuli source 540 each other. In an embodiment, the insulation pattern 544 insulates elements requiring electric insulation such as the source S, the drain D, the gate 520, and the conduction pattern 552.

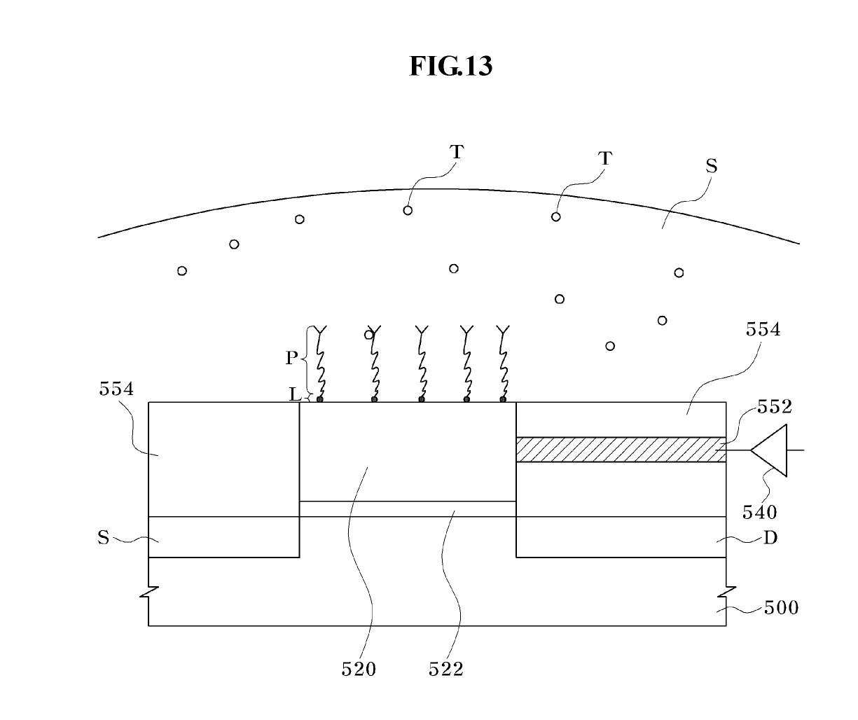

FIG. 13 is a view showing a solution containing target material Ts on the biosensor according to the embodiment. The biosensor according to the embodiment is described under the assumption that the probe P has negative charge and the stimuli source applies a step pulse having positive potential.

Referring to FIGS. 13 and 10, before measuring the concentration of a target material, using the biosensor according to the embodiment, it is preferable to bind the probe P with the target material, for example, by using the binding enhancing apparatus or performing the method for binding enhancement according to an embodiment of the present invention. When target materials T to be sensed are in an electrolyte solution such as blood, the sensing sensitivity is decreased by the charge screening effect, as described above. This is because, as shown in FIG. 10a, the probe P and the target material T bound to the probe P have charge and the ions having opposite charge in the electrolyte solution surround the target material, such that the opposite charge is not induced to the channel unit by the target material.

FIG. 10b is a schematic view showing that status at t=0+1, right after a stimulus source applies a step waveform. Referring to FIGS. 12 and 10b, the stimuli source applies an electric stimulus having a step wave through the conduction pattern and the gate. After the stimuli source applies a step waveform, the probe P is contracted and an EDL (Electrical Double Layer) is expanded by the applied positive potential. Therefore, the electric field travels to the extension region of the EDL without being shield, such that it influences the probe P and the positive ions around the probe P.

Referring to FIGS. 13 and 10c, the electric field penetrates into it while the EDL expands in a transient period after t=0+1, such that the positive ions around the probe P and the target material are repelled away. Accordingly, the electric field flux by the target material T travel to the gate without being blocked by the surround positive ions, and induce charge to the gate. Therefore, the threshold voltage of a MOS transistor is changed by the charge induced to the gate. By sensing the change of the threshold voltage of the MOS transistor, it is possible to measure the concentration of the target materials in the solution.

Referring to FIG. 10d, the EDL that has expanded is redistributed onto the surface of the channel and shields the electric field in the steady state after the transient period at t>0, such that the positive ions that has been repelled away are attracted by the positive charge of the probe P and the target material and surround again the probe P.

In the embodiment, it is exemplified that the probe P and the target material T have negative charge and the stimuli source applies a step waveform having positive potential shown in FIG. 9a, the probe P and the target material T may have negative charge and the stimuli source may apply a step waveform having negative potential shown in FIG. 9b. Even though negative potential is applied, the EDL expands and the electric field penetrates into the expansion region of the EDL, such that the ions close to the probe P and the target material T can be repelled away from the probe P and the target material T. Accordingly, it is possible to measure the charge induced to the channel unit in the transient period until the EDL is redistributed after the ions are repelled away from the probe P and the target material T.

Further, when the probe P and the target material T have positive charge, the stimuli source may apply a step waveform having negative potential, as shown in FIG. 9b. Further, after any one of the pulses shown in FIG. 6 is applied, sensing can be performed in the corresponding transient period. However, this is apparent to those skilled in the art and is not described for brief and clear description.

In an embodiment of the biosensor according to the present invention, sensing is performed in the transient period, after an electric stimulus is applied. FIG. 11 is a view showing a transient period of an electric stimulus applied by the stimuli source. Referring to FIG. 11, an electric stimulus is applied and then sensing is performed in the transient period. The transient period means a state until an EDL expands after a stimulus is applied, as described above, the ions around the probe P and the target T are moved by the penetrating electric field and the ions surround again the probe P and the target material T by the EDL redistributed and shielding the electric field. Accordingly, the duration time of the transient period depends on the structure of the biosensor and the concentration of the ions around the probe P and the target material T too. That is, the transient period decreases, when the concentration of the ions is high, whereas the transient period increases, when the concentration of the ions is low.

In general, for a buffer solution containing sodium chloride (NaCl) use for sensing of a biomaterial, the period from the point of time of applying potential to O(10̂(−6)) sec where an EDL is redistributed can be considered as the transient period, and to 0−O(10̂(−2)) sec when it is measured in water with impurities refined at a predetermined degree. Further, when other ions are refined at higher degree of refining on a predetermine measuring platform than the state described above, the transient period may increase up to 500 msec, and the transient period may decrease up to 0.1 nsec, for a buffer solution containing NaCl with high concentration.

As an embodiment, a buffer solution of PBS (Phosphate Buffered Saline) or TE (Tris-EDTA) may be used for sensing a biomaterial. When ions are contained with high concentration in the PBS or TE buffer solution, the transient period may be about 0.1 nsec when a target material is sensed, whereas the transient period may be about 500 msec when the ions are contained with low concentration.

For example, referring to FIG. 11a, when a stimuli source applies a stimulus having a step waveform of positive potential, sensing is performed from the rising edge up to a maximum of 500 msec (tsense). As another example, referring to FIG. 11b, when a stimuli source applies a stimulus having a step waveform of negative potential, sensing is performed from the falling edge up to a maximum of 500 msec (tsense). As another example, referring to FIG. 11c, when a stimuli source applies a stimulus having a rectangular pulse waveform, sensing is performed in the section from the rising edge t0 to 500 mse (tsense) and/or sensing is performed in the range from the falling edge t1 to 500 msec (tsense). As another example, referring to FIG. 11d, when a stimuli source applies an electric stimulus having a sine waveform, sensing is performed in the section from the end of the rising edge t2 to 500 mse (tsense) and/or sensing is performed in the range from the end of the falling edge t3 to 500 msec (tsense). However, the figures shows the sections where sensing is performed, sensing may keep performed for the sensing section (tsense), and the sensing may be performed one time or more in the corresponding sections. Further, when a continuous pulse is applied, as in FIGS. 11c and 11d, sensing may be performed in the section from the rising edge and/or the falling edge to 500 msec.

However, sensing for 500 msec shown in the figures means sensing within 500 msec after a stimulus is applied, which does not means that 500 msec is necessary or sensing should be performed for 500 msec, and sensing may be performed in any one section for 500 msec after a stimulus is applied.

Accordingly, the section where a stimulus is applied and sensing is performed depends on the concentration of the ions in the solution or the concentration of the target material, such that the transient period may be about 0.1 nsec, in which the frequency of the applied stimulus may be a maximum of 10 GHz. Further, the transient period may be 500 msec, in which the frequency of the applied stimulus may be a maximum of 2 Hz.

An exemplary embodiment of a sensing method according to the present invention is described hereafter with reference to the accompanying drawings. A sensing method according to an embodiment of the present invention includes: preparing a biosensor including at least one probe P that is complementarily bound with a target material; positioning a solution containing target materials that is complementarily bound with the probe P onto the biosensor; applying an electric stimulus by means of a stimuli source; and sensing the target materials in a transient period of the applied electric stimulus. The part overlapping the embodiment described above may not be described to make the description brief and clear.

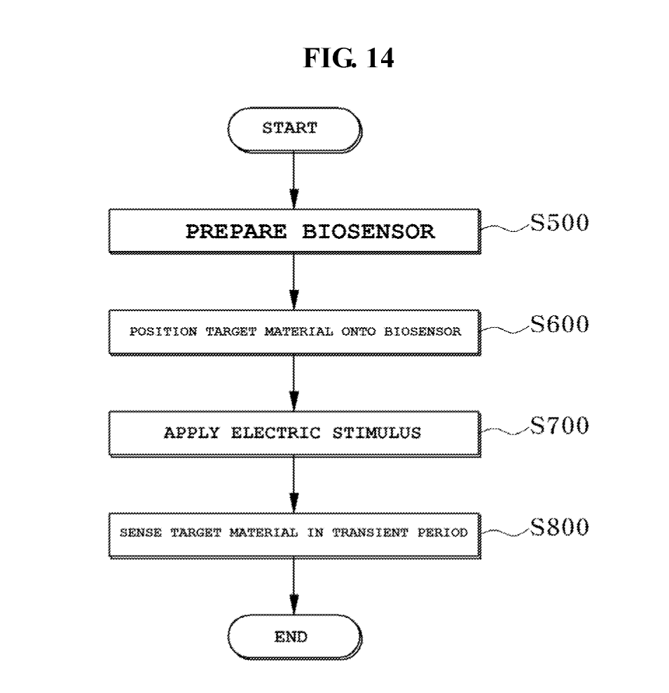

FIG. 14 is a flowchart illustrating an embodiment of a sensing method according to the present invention. Referring to FIG. 14, a biosensor containing at least one probe P that is complementarily bound with a target material is prepared (S500). As another embodiment, the biosensor may be formed in an array, as shown in FIGS. 2A and 2B, and may be formed in any types of arrays, which are not shown in the figures. As another embodiment, the biosensor has a first electrode and a second electrode, as in FIG. 8, and may include a probe P on the channel unit 320 connecting the first electrode 300 and the second electrode 310 each other. As another embodiment, the biosensor has a source S, a drain D, and a gate, as in FIG. 11, and may include a probe P on the gate.

A solution containing target materials is positioned onto biosensor (S200). The material of the probe P in the bio sensor may have predetermined charge, the probe P is in a relaxation state, when an electric stimulus is not applied by a stimuli source, and the target materials are uniformly distributed in the solution. Accordingly, the binding ratio per unit time at which the relaxed probe P and the target material are bound is low, and particularly, when the mobility or the concentration of the target materials is low, the binding ratio per unit time of the probe P and the target material further decreases.

A stimuli source applies an electric stimulus (S300). In an embodiment, the electric stimulus applied by the stimuli source may be a step waveform having positive potential, as shown in FIG. 9a. In another embodiment, the electric stimulus applied by the stimuli source may be a step waveform having negative potential shown in FIG. 9b. In another embodiment, the electric stimulus applied by the stimuli source may be at least one of a rectangular pulse, a triangular pulse, a sinusoidal pulse, and a linear superposition of them or combination of them in time sequence, as shown in FIG. 6. In another embodiment, though not shown in the figures, the stimuli source may apply an electric stimulus while alternating a section where an electric stimulus is applied and a predetermined suspension period.

In this step, since the positive ions and the negative ions in the EDL are randomly mixed while being redistributed by the electric filed due to the potential from the stimuli source, by the electric stimulus from the stimuli source, the electric field penetrates into the expansion region of the EDL without being shielded and influences the probe P and the ions around the probe P.

In an embodiment, when the swing range of the applied electric stimulus is too big, or the electric stimulus has a low frequency or is continuously applied, the solution containing target materials may be electrolyzed or the probe P and the target materials may be denatured. Accordingly, it is required to apply an electric stimulus having appropriate swing range and frequency in order to prevent the denaturation or electrolysis, and to appropriately control the duration period of the electric stimulus. For example, as for the swing range of an electric stimulus, an electric stimulus that swings within the range of −10V to 10V is applied. For example, as for the frequency of an electric stimulus, an electric stimulus having a frequency within 10 GHz, more than zero, is applied. For example, as for the duration time of an electric stimulus, an electric stimulus is applied within one hour, more than zero.

The target material is sensed in the transient period of the applied electric stimulus (S400). In an embodiment, the electric field penetrates into the EDL while the EDL expands in a transient period, such that the ions around the probe P and the target material are repelled away. Accordingly, the electric field flux by the target material travel to the channel unit 530 and induce charge to the channel unit without being blocked by the surrounding ions and the read-out circuit unit can measure the concentration of the target materials in the solution by sensing the induced charge.

Therefore, sensing is supposed to be performed in the transient period with the ions, which has been around the probe P and the target materials, repelled away by the applied electric field. An embodiment of performing the sensing was described above with reference to FIG. 11.

Although the present invention has been described in connection with the embodiments shown in the drawings in order to help understand the present invention, the embodiments are only examples and it should be understood that various changes and equivalent modifications can be implemented from the present invention by those skilled in the art. Therefore, the technical protection range of the present invention should be determined by the accompanying claims.

What is claimed is:

1. A biosensor comprising:
a first electrode;
a second electrode spaced from the first electrode;
a channel unit electrically connected at a portion with the first electrode and electrically connected at another portion with the second electrode;
a stimuli source electrically connected with the channel unit and applying an electric stimulus; and
probes connected to the channel unit and complementarily bound with target materials to sense.

2. The biosensor of claim 1, wherein the channel unit is formed by one channel sub-unit or by electrically connecting one or more channel sub-units.

3. The biosensor of claim 2, wherein the channel sub-unit includes at least one of a semiconductor, a nano structure, and metal.

4. The biosensor of claim 1, wherein the target materials are contained in a solution.

5. The biosensor of claim 1, wherein any one of the first electrode and the second electrode is electrically connected with the stimuli source.

6. The biosensor of claim 1, wherein the stimuli source applies a pulse.

7. The biosensor of claim 6, wherein the pulse is a step pulse.

8. The biosensor of claim 6, wherein the pulse is a rectangular pulse, a triangular pulse, a sinusoidal pulse, a superposition of at least one of the pulses, and a pulse having at least one of the pulses in time sequence.

9. The biosensor of claim 1, wherein the first electrode surrounds the second electrode.

10. The biosensor of claim 1, wherein a plurality of biosensors is formed in an array and the first electrodes are electrically connected each other.

11. The biosensor of claim 1, which senses the target materials in a transient period to the application of the electric stimulus.

12. The biosensor of claim 11, wherein the transient period is within 500 ms after a rising edge or a falling edge of the electric stimulus.

13. A sensing method comprising:
preparing a biosensor including at least one probe that is complementarily bound with a target material;
positioning a solution containing target materials that is complementarily bound with the probe onto the biosensor;
applying an electric stimulus by means of a stimuli source; and
sensing the target materials in a transient period of the applied electric stimulus.

14. The method of claim 13, wherein the applying of an electric stimulus applies a step pulse.

15. The method of claim 13, wherein the applying of an electric stimulus applies at least one of a rectangular pulse, a triangular pulse, a sinusoidal pulse, a superposition of at least one of the pulses, and a pulse having the pulses in time sequence.

16. The method of claim 13, wherein the applying of an electric stimulus applies an electric stimulus that swings within the range of −10V to 10V.

17. The method of claim 13, wherein the applying of an electric stimulus applies an electric stimulus having a frequency within 10 GHz, more than zero.

18. The method of claim 13, wherein the applying of an electric stimulus is performed within one hour, more than zero.

19. The method of claim 13, wherein the sensing in a transient period is performed within 500 msec after a rising edge of the electric stimulus.

20. The method of claim 13, wherein the sensing in a transient period is performed within 500 msec after a falling edge of the electric stimulus.
